(19) United States
(12) Patent Application Publication
(10) Pub. No.: US 2019/0025283 A1
(43) Pub. Date: Jan. 24, 2019

(54) A UREA, PHOSPHATE AND PH MEASURING DEVICE

(71) Applicants: Ibrahim ISILDAK, Esenler/Istanbul (TR); Ozlem TAVUKCUOGLU, Kadikoy/Istanbul (TR); Mustafa NIGDE, Pendik/Istanbul (TR); Ridvan YILDIRIM, Pendik/Istanbul (TR); Ismail AGIR, Bahcelievler/Istanbul (TR)

(72) Inventors: Ibrahim ISILDAK, Esenler/Istanbul (TR); Ozlem TAVUKCUOGLU, Kadikoy/Istanbul (TR); Mustafa NIGDE, Pendik/Istanbul (TR); Ridvan YILDIRIM, Pendik/Istanbul (TR); Ismail AGIR, Bahcelievler/Istanbul (TR)

(21) Appl. No.: 15/757,428

(22) PCT Filed: Aug. 29, 2016
(86) PCT No.: PCT/TR2016/050313

§ 371 Date: Mar. 5, 2018

(30) Foreign Application Priority Data
Sep. 7, 2015 (TR) 2015/11075

(51) Int. Cl. G01N 33/49; A61B 5/145; A61B 5/1486; A61B 5/1468

(52) U.S. Cl. CPC G01N 33/492; A61B 5/14539; A61B 5/14546; A61B 5/1495; A61B 5/1468; A61B 2505/07; A61B 2562/0295; A61B 5/1486

(57) ABSTRACT

A potentiometric measurement device that can be carried by the patient or user easily and it can measure the urea, phosphate and pH values in an amount of blood in every environment, such as home, office and hospital. It includes a measurement module, a control module and a communication unit. It includes at least one urea sensing unit for determination of the urea amount in blood, at least one phosphate sensing unit for determination of the phosphate amount in blood, at least one pH sensing unit for determination of the hydronium ion level in blood. It includes a control module that determines urea, phosphate and pH values in blood via the data measured by the urea sensing unit, phosphate sensing unit and pH sensing unit.

### RELATED TECHNICAL FIELD OF THE INVENTION

This invention is related with a device which measures urea, phosphate and pH values in a little amount of blood at the same time.

### PRIOR ART

The chronic renal failure is a clinical disease which appears as a result of loss of nephrons progressively and irremediably due to a reason related or not related with kidneys. Liquid, acid-base and electrolyte balance disorders are frequent problems in the renal failure. In case of high level loss of kidney functions; to remove phosphate and urea from the body fluid cannot be provided and dialysis is required. Therefore; frequent monitoring of urea, phosphate (PO₄³⁻) and pH parameters in blood through many years has a significant importance in order to determine the levels of uremia, hyperphosphatemia and acid-base balance disorder in the renal failure patients. Providing diet according to urea, phosphate and pH values via monitoring these values in the blood of patient people who are in the dialysis process or who does not need dialysis yet, but has reduced kidney functions can minimize long term negative effects which occur because of the disease in these patients.

Nowadays, the dialysis patients need to go to hospitals or dialysis centers in order to learn the urea, phosphate and pH values in their blood.

Determination of urea, phosphate and pH is performed generally via desktop systems in hospitals and dialysis centers. This situation requires that patients go to the hospital or dialysis center periodically and affects the life quality of the patient negatively. As a result of these measurements which are performed frequently, dialysis patients can live more comfortably by controlling their diet.

In addition, blood samples in high volumes are generally taken from patients via invasive application from vascular access for urea, phosphate and pH measurements in said methods used in the state of the art.

Nowadays, there is not any system or device from which patients can learn their said values without depending on a hospital, laboratory or dialysis center. Blood urea value is measured via spectrophotometric, enzymatic and kinetic methods; phosphate value is measured via the spectrophotometric method; pH value is measured via desktop systems with special electrodes in blood gas analyzers.

At the present time, another method which can be used for determination of different analytes is the potentiometric method. Potentiometry is a determination method in which the changing potential difference between the ion selective sensor and reference electrode is measured depending on the activities of the ions in the medium in case that there is no current or very little current passing through the solution in a electrochemical cell.

The ion concentration is measured directly by considering the changing potential of the ion selective sensor in the potentiometry. The potentiometric system consists of a test cell (electrolytic solution), ion selective sensor or molecule selective biosensor (variable potential) which is connected to it, reference electrode (constant potential) and a stable potentiometer.

An ion selective sensor works together with at least one reference electrode in the potentiometric measurements.

In the patent document with the number of U.S. Pat. No. 8,273,228B2 at the state of the art, a portable device which provides measurement and control by using biosensors in the determination of glucose and other analytes in the biological fluids is described.

In the patent document with the number of U.S. Pat. No. 5,711,862 at the state of the art, a biochemical enzyme sensor which measures the glucose value voltametrically is described.

In the patent document with the number of US2006040251 at the state of the art, a device which can measure one single agent like glucose via the voltametric method is described.

### BRIEF DESCRIPTION OF THE INVENTION

The aim of this invention is a urea, phosphate and pH measuring device which can perform the measurement of urea, phosphate and pH values in blood at the same time with a little amount of blood sample.

The disclosed measurement device of the invention can be carried easily by the patient or user and can measure urea, phosphate and pH values in a little amount of blood taken from finger in every environment.

The disclosed measurement device of the invention comprises at least one control module, at least one measurement division and at least one communication module.

The control module is a unit which determines the urea, phosphate and pH value in blood simultaneously and correctly and provides its presentation to the user by comparing the values determined and the reference values.

The control module is a module which presents to the user if the measurement values which it takes from the urea sensing unit, phosphate sensing unit and pH sensing unit are in the interval that healthy humans need to have by comparing said data with the reference values and how much they are lower or higher if they are not in the value interval that healthy humans need to have by controlling them.

The control module provides that emergency information is given to the user in case that the values measured are lower or higher than the value interval of healthy humans.

The measurement module comprises at least one urea sensing unit for determining urea amount in blood, at least one phosphate sensing unit for determining phosphate amount in blood, at least one pH sensing unit for determining hydronium ion level in blood and a reference electrode unit.

The measurement module is in the “strip” form comprising at least one urea sensing unit, at least one phosphate sensing unit and at least one pH sensing unit.

The strip comprises a sample division in which the blood sample is positioned.

The sample division comprises wettable polymer, a hydrogel or cellulosic filtration layer which provide filtration of the blood sample positioned.

The filtration layer comprises a solution which performs calibration for measurements of urea, phosphate and pH.

The solution consists of the components whose urea, phosphate and pH levels are known.

The urea sensing unit comprises at least one urea biosensor which has the urease enzyme for determination of urea amount in blood.

The urea biosensor detects ammonium ion changes which occur as a result of hydrolysis reaction catalyzed by urease.

The phosphate sensing unit comprises at least one phosphate sensor which is in composite structure comprising an ionophore which is sensitive for HPO₄²⁻ (hydrogen phosphate) ion, epoxy, hardener and modified graphite or graphene for determining phosphate amount in blood.

pH sensing unit comprises at least one composite structured pH sensor which can measure pH value for determination acidity or alkalinity of the patient blood, which is necessary for correction of urea and phosphate amounts at the same time.

The reference unit comprises at least one solid-state reference electrode which shows constant potential in the sample solution for determination of urea, phosphate concentrations and pH in the blood of patient.

The communication module is the module which can provide remote access via a smart device, can also conduct the transmission of data obtained in consequence of the measurements to the patient remotely via wireless connections.

The communication module comprises the unit that provides transmission of data which is received from the measurement module and whose comparison and calculation is performed by the control unit to the patient and/or doctor.

The communication module comprises a sim card which provides transmission of data obtained in consequence of the measurement to the patient and/or doctor.

The disclosed measurement device of the invention comprises a display which shows the values obtained in consequence of the measurement to the patient.

Due to the disclosed measurement device of the invention; the patients who receives dialysis treatment can monitor the urea, phosphate and pH values with a little volume of blood sample which they take from finger on their own without going to hospitals or dialysis centers.

In addition, the disclosed measurement device of the invention can be applied to the dialysis patients also by health officers in hospitals easily. It provides to get result faster and more practical than the desktop measurement systems used in the processes before the disclosed device of the invention.

Additionally, it is not necessary to enter to the vascular access via needle for determination of the levels of urea, phosphate and pH agents in the patient blood due to the disclosed device of the invention. The life quality of patients is increased by providing them not to suffer due to a little amount of blood sample taken from finger.

In addition to patients' own monitoring of said agents in their blood via the disclosed urea, phosphate and pH measuring device of the invention; the results obtained can be sent to the related doctor and that's why, time and duration of dialysis can be decided earlier effectively.

Hyperphosphatemia and uremic syndromes which occur in the chronic renal failure disease may cause life threat for these patients quickly. Slowing down of threat can be provided via dialysis and diet applications. Therefore, to follow the patients who have chronic renal failure closely has importance. This situation requires that urea and phosphate blood values which cause uremia and phosphatemia are measured frequently. That's why, the disclosed measurement device of the invention provides that blood urea, phosphate levels of the patients who have a chronic renal failure threat and in addition, blood pH level which is closely related to these parameters are measured effectively.

The disclosed measurement device of the invention can be applied easily and comfortably by the patient at home with a high originality, sensitivity and accuracy percentage for monitoring uremic syndrome and hyperphosphatemia in renal failure patients effectively.

Due to the disclosed urea, phosphate and pH measurement device of the invention; the patients can determine their diet and treatment regimen on their own or with the doctor deliberatively, not randomly, by considering levels of these agents which they monitor.

### DETAILED DESCRIPTION OF THE INVENTION

### Description of the Figures

FIG. 1—It is the view of the disclosed measurement device of the invention.

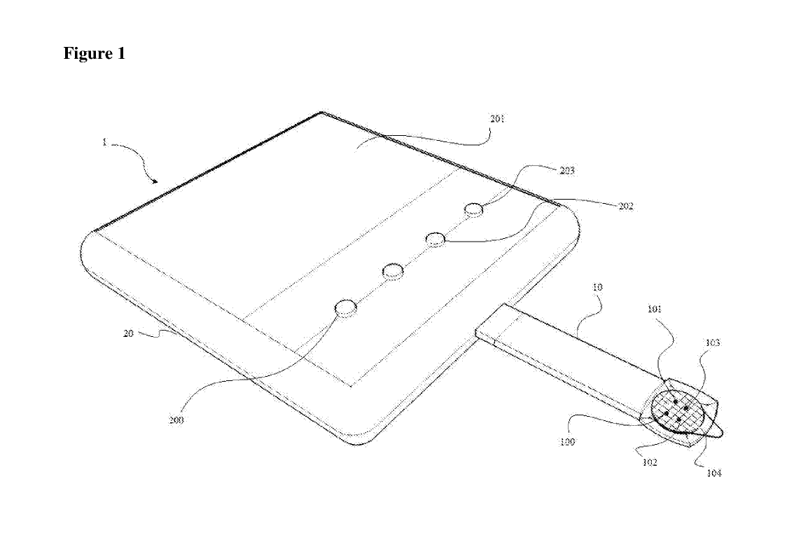

FIG. 2—It is the view of the disclosed measurement device of the invention during its application.

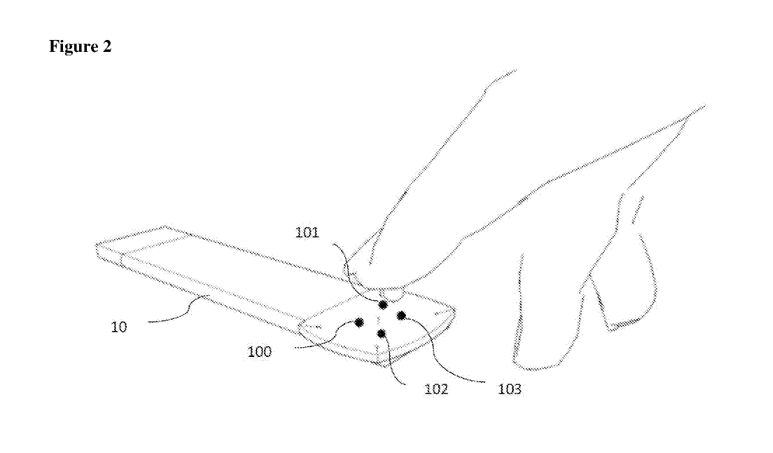

FIG. 3—It is the view of the strip type measurement module of the disclosed measurement device of the invention.

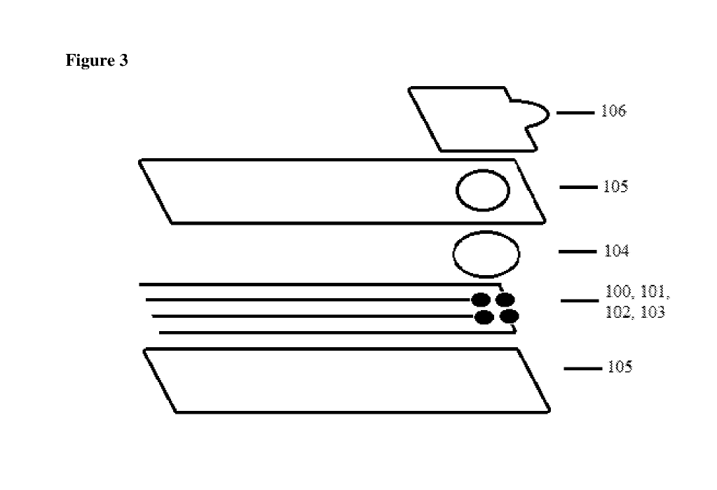

FIG. 4—It is the block diagram of the disclosed device of the invention.

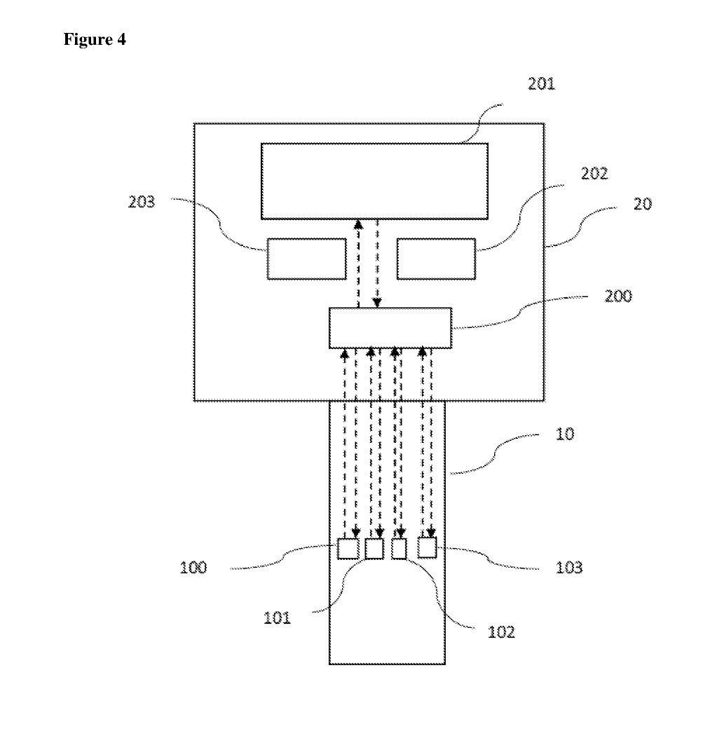

### DESCRIPTION OF THE REFERENCES IN THE FIGURES

The parts in the figures are numbered respectively and the equivalents of these numbers are given below.

1. Urea, phosphate and pH measuring device

10. Measurement module
100. Urea sensing unit
101. Phosphate sensing unit
102. pH sensing unit
103. Reference unit
104. Sampling and filtration unit
105. Plastic layer
106. Removable plastic cover

20. Control module
200. Control unit
201. Display
202. Power unit

30. Communication module

The disclosed measurement device (1) of the invention comprises a measurement module (10) in which urea, phosphate and pH measurements are performed in the blood taken from the patient, a control module (20) in which said results are calculated and shown to the patient and a communication module (30) which can provide remote access.

The disclosed measurement device (1) of the invention works potentiometrically.

The measurement module (10) is in the strip form.

The strip shaped measurement module (10) comprises at least one urea sensing unit (100) which performs urea measurement, at least one phosphate sensing unit (101) which performs phosphate measurement and at least one pH sensing unit (102) which performs pH measurement.

The urea sensing unit (100) comprises an entire solid-state contact ammonium selective sensor. There is the urease enzyme as a biolayer on the sensor surface. Urease is an enzyme which is special for urea. The urease enzyme releases ammonium ion to the environment by giving reaction with urea. The urea sensing unit (100) provides determination of the urea concentration in blood by finding the concentration of ammonium ion released. In another embodiment of the invention, the urea sensing unit (100) can perform the ammonium measurement process also via another enzyme.

In the embodiment of the invention, the urea sensing unit (100) comprises at least one urea biosensor which provides determination of urea for finding the urea amount in blood.

The phosphate sensing unit (101) comprises a special composite material comprising a phosphate complex which is sensitive to HPO₄²⁻ ion. Determination of the phosphate concentration in blood is provided via determination of the concentration of this ion.

In the embodiment of the invention, the phosphate sensing unit (101) comprises at least one phosphate sensor for determining the amount of phosphate ion in blood.

pH sensing unit (102) comprises a special composite material comprising quinhydron which is sensitive to H₃O⁺ ion. pH sensing unit (102) comprises at least one pH sensor for determination of the pH of blood in the embodiment of the invention.

The strip shaped measurement module (10) also comprises a reference unit (103). The reference unit (103) comprises at least one Ag/AgCl based composite structured reference electrode used in providing urea, phosphate and pH measurements in blood.

In an embodiment of the invention, reference electrode does not participate in the measurements. It acts as reference by producing a constant potential in the blood sample for performing urea, phosphate and pH measurements correctly.

In the disclosed device (1) of the invention performing urea, phosphate and pH measurements, only one reference electrode which is connected in series with the urea, phosphate and pH sensors works.

The measurement module (10) comprises a sampling and filtration unit (104) which the blood sample whose urea, phosphate and pH values are to be measured is dropped into. It is provided that the filtration unit absorbs a calibration solution which provides sensors to measure reliably.

The measurement module (10) comprises two plastic layers (105).

The measurement module (10) comprises a plastic cover (106) which can be removed and thrown away. The removable plastic cover (106) is to provide conservation of the calibration solution absorbed into the sampling and filtration unit (104).

In an embodiment of the invention; the urea sensing unit (100), phosphate sensing unit (101), pH sensing unit (102), reference unit (103) and sampling and filtration unit (104) are positioned between two plastic layers (105).

In an embodiment of the invention; the urea sensing unit (100), phosphate sensing unit (101), pH sensing unit (102) and reference unit (103) are positioned at the lower side of the sampling and filtration unit (104).

In an embodiment of the invention; one removable plastic cover (106), urea sensing unit (100), phosphate sensing unit (101), pH sensing unit (102) and sampling and filtration unit (104) are positioned between two plastic layers (105).

Two plastic layers (105) provide insulation. However, the part at the side of sampling and filtration unit (104) is left open.

The removable plastic cover (106) is designed as it acts like a cap on this part whose top is open. Before the measurement process with the blood sample is started, it is removed and thrown away.

The sensing units (100, 101 and 102) perform reading from a little amount of blood sample which is taken from finger and dropped onto the sampling and filtration unit (104).

The sensing units (100, 101 and 102) which are to perform said measurements comprise polymer, ionophore, plasticizer, modified graphite, modified graphene, ionic resistance reducer and polymeric membrane which has selectivity increasing materials and composite membrane structures.

The disclosed urea, phosphate and pH measuring device (1) of the invention comprises a control module (20) in which the data received in consequence of the measurement from the blood sample is processed.

The control module (20) comprises a control unit (200) which evaluates the data that it receives from the urea sensing unit (100) determining the urea value in blood, from the phosphate sensing unit (101) determining the phosphate value and from the pH sensing unit (102) determining pH value; a display (201) which shows the values to the patient or user and a power unit (202). The power unit (202) may also be added from outside according to the embodiment of the invention.

The control module (20) comprises a control unit (200) which provides that emergency information is given to the user if the values measured are lower or higher in a predetermined amount than the value interval that healthy humans need to have.

The control unit (200) compares the data that it receives from the urea sensing unit (100), phosphate sensing unit (101) and pH sensing unit (102) with the reference values and transmits to the user if the urea, phosphate and pH values in his blood is in a normal value interval.

The normal values mean the urea, phosphate amount that healthy humans need to have in their blood and the pH of the blood of healthy humans.

For example; the phosphate level that needs to be in normal human blood is between 2.5-4.8 mg/dl. If the phosphate value is calculated by the control unit (200) at said interval in consequence of the measurement, the disclosed device (1) of the invention will transmit that the phosphate value is normal to the patient via the display (201).

In an embodiment of the invention; the control unit (200) comprises a micro processor or micro controller unit, an analog to digital converter (ADC), a channel multiplexor (MUX), an operational amplifier (Op-Amp).

Connecting the sensing units (100, 101 and 102) which have very high impedance to the ADC integrated circuits which have a low input impedance is provided via the control unit (200).

Output signals of the sensing units (100, 101 and 102) which are converted to a digital magnitude are sent to the control module (20) via a transmitter module.

In an embodiment of the invention, the control module (20) can connect to a smart device via bluetooth wirelessly.

The display (201) is a screen that provides transmission of the data to the user. The user can make all necessary adjustments by using this screen and monitor the data via menus on the screen.

The power unit (202) is a unit which provides the necessary energy for working of the urea, phosphate and pH measuring device (1).

The disclosed urea, phosphate and pH measuring device (1) of the invention comprises a communication module (30) which the data received from blood sample in consequence of the measurement is processed into.

The communication module (30) provides transmission of the data obtained in consequence of the measurement to the patient and/or related doctor.

In an embodiment of the invention, the communication unit (203) comprises a sim card which provides the transmission of the results to the patient and/or doctor.

The disclosed urea, phosphate and pH measuring device (1) of the invention has a size such that it can be carried easily.

The measurement device (1) monitors also the pH value together with the phosphate and urea amount. In an embodiment of the invention; urea, phosphate and pH measurement in 5-10 microliters of a blood sample is performed via the urea sensing unit (100), phosphate sensing unit (101) and pH sensing unit (102) in the strip type measurement module (10).

Therefore; a safe and effective monitoring which can be used by the patient at home easily and has high originality, sensitivity and accuracy percentage is expected in early detection of the uremic syndrome and hyperphosphatemia in renal failure patients.

The invention is not limited with the disclosed embodiments above, a skilled person in the art can produce different embodiments of the invention easily. They should be evaluated within the scope of invention protection demanded with claims.

1-18. (canceled)

19. A potentiometric measurement device (1) comprising a measurement module (10) and a reference unit (103) and characterized by at least one urea sensing unit (100) for determining the urea amount in blood correctly, at least one phosphate sensing unit (101) for determining the phosphate amount in blood, at least one pH sensing unit (102) for determining the hydronium ion level in blood, and at least one sampling and filtration unit (104) which the blood sample whose urea, phosphate and pH values are to be measured is dropped into, wherein the urea sensing unit (100), phosphate sensing unit (101), pH sensing unit (102) and reference unit (103) are positioned at the lower side of the sampling and filtration unit (104) and wherein reference unit (103) comprises only one reference electrode showing constant potential in the sample solution for determination of the urea, phosphate concentration in the blood of patient and the pH of the blood.

20. A potentiometric measurement device (1) such that it can be carried easily by the patient or user and can measure the urea, phosphate and pH values in an amount of blood at every environment like home, office, hospital; characterized by comprising a control module (20) which determines the urea, phosphate and pH value in blood dropped into a sampling and filtration unit (104), via the data measured by the urea sensing unit (100), phosphate sensing unit (101), pH sensing unit (102) and a reference unit (103) comprising only one reference electrode showing constant potential in the sample solution for determination of the urea, phosphate concentration in the blood of patient and the pH of the blood, and provides comparing said data with the reference values of healthy humans and presentation to the user.

21. A measurement device (1) according to claim 19 characterized by comprising a measurement module (10) which can measure the urea, phosphate and pH value from 5-10 microliters of blood such that it can be taken from a finger tip.

22. A measurement device (1) according to claim 19 characterized by comprising a control module (20) which determines the urea, phosphate and pH value in blood at the same time and provides comparing said data with reference values and presentation to the user at the same time.

23. A measurement device (1) according to claim 21 characterized by comprising the control module (20) which has a control unit (200) that presents to the user if the measurement values which it takes from the urea sensing unit (100), phosphate sensing unit (101) and pH sensing unit (102) are in the interval that healthy humans need to have by comparing them with the reference values and how much they are lower or higher if they are not in the value interval that healthy humans need to have by controlling them.

24. A measurement device (1) according to claim 22 characterized by comprising the control module (20) having the control unit (200) which provides that emergency information is given to the user if the values measured are lower or higher in a predetermined amount than the value interval of healthy humans.

25. A measurement device (1) according to claim 24 characterized by comprising a measurement module (10) which comprises at least one urea sensing unit (100), at least one phosphate sensing unit (101), at least one pH sensing unit (102) and at least one reference unit (103).

26. A measurement device (1) according to claim 25 characterized by comprising the measurement module (10) which comprises a strip type sampling and filtration unit (104) which a calibration solution is provided to be absorbed by and in which the blood sample is to be positioned.

27. A measurement device (1) according to claim 24 characterized by comprising at least one urea sensing unit (100) which provides determination of the urea concentration in blood by sensing the changes of the ammonium ion that is formed as a result of the urease catalyzed hydrolysis reaction.

28. A measurement device (1) according to claim 26 characterized by comprising the urea sensing unit (100) which comprises at least one urea biosensor having the urease enzyme for determination of the urea amount in blood.

29. A measurement device (1) according to claim 27 characterized by comprising the phosphate sensing unit (101) which is sensitive to HPO4²⁻ (hydrogen phosphate) ion for determination of the phosphate amount in blood.

30. A measurement device (1) according to claim 28 characterized by comprising the phosphate sensing unit (101) which has at least one composite structured sensor comprising ionophore that is sensitive to HPO4²⁻ (hydrogen phosphate), epoxy, hardener and modified graphite or graphene for determination of the phosphate amount in blood.

31. A measurement device (1) according to claim 29 characterized by comprising at least one pH sensing unit (102) which can measure the pH value for determination of acidity or alkalinity of the patient's blood, is necessary for correction of the urea and phosphate amounts at the same time.

32. A measurement device (1) according to claim 31 characterized by comprising a reference unit (103) which comprises at least one reference electrode connected in series with the urea sensing unit (100), phosphate sensing unit (101) and pH sensing unit (102) such that it provides to be in size that it can be carried easily.

33. A measurement device (1) according to claim 19 characterized by comprising a communication unit (203) which provides transmission of the data obtained in consequence of the measurements to the patient and/or doctor wirelessly by providing remote access via a smart device after the data received from the measurement module (10) are evaluated and calculated by the control module (20).

34. A measurement device (1) according to claim 32 characterized by comprising the communication unit (203) which comprises a sim card providing transmission of the data obtained in consequence of the measurement to the patient and/or doctor.

35. A measurement device (1) according to claim 19 characterized by comprising a display (201) which shows the interpreted values obtained in consequence of the measurement to the patient and/or doctor.
